(19) United States
(12) Patent Application Publication
(10) Pub. No.: US 2019/0313921 A1
(43) Pub. Date: Oct. 17, 2019

(54) MEASUREMENT DEVICE, MEASUREMENT METHOD, AND TRANSITORY COMPUTER READABLE MEDIUM

(71) Applicants: OMRON HEALTHCARE Co., Ltd., Kyoto (JP); A SCHOOL CORPORATION KANSAI UNIVERSITY, Osaka (JP); PUBLIC UNIVERSITY CORPORATION NARA MEDICAL UNIVERSITY, Nara (JP)

(72) Inventors: Miki IMAMURA, Kyoto (JP); Yukiya SAWANOI, Kyoto (JP); Toshihiko OGURA, Kyoto (JP); Hideo UTSUNO, Osaka (JP); Kimihiko KICHIKAWA, Nara (JP); Shigeo ICHIHASHI, Nara (JP); Shinichi IWAKOSHI, Nara (JP)

(73) Assignees: OMRON HEALTHCARE Co., Ltd., Kyoto (JP); A SCHOOL CORPORATION KANSAI UNIVERSITY, Osaka (JP); PUBLIC UNIVERSITY CORPORATION NARA MEDICAL UNIVERSITY, Nara (JP)

(21) Appl. No.: 16/452,764

(22) Filed: Jun. 26, 2019

Related U.S. Application Data
(63) Continuation of application No. PCT/JP2017/036539, filed on Oct. 6, 2017.

(30) Foreign Application Priority Data
Dec. 27, 2016 (JP) 2016-252951

(51) Int. Cl. A61B 5/02; A61B 5/024; A61B 5/00; G16H 50/20

(52) U.S. Cl. CPC A61B 5/02014; A61B 5/02438; G16H 50/20; A61B 5/6829; A61B 5/7282; A61B 5/6824

(57) ABSTRACT

A measurement device includes a pulse wave signal acquisition unit, a pulse wave velocity calculator, a transfer function calculator, a phase diagram classifier, and an aneurysm determinator. The pulse wave signal acquisition unit acquires time-series pulse wave signals of each of an upper arm and an ankle of the subject. The pulse wave velocity calculator obtains a brachial-ankle pulse wave velocity based on the pulse wave signal of the upper arm and the pulse wave signal of the ankle. The transfer function calculator calculates a transfer function from the pulse wave signal of the upper arm and the pulse wave signal of the ankle. The phase diagram classifier classifies the phase diagram of each subject into any one of four groups. The aneurysm determinator determines presence or absence of the abdominal aortic aneurysm by a criterion set according to each group.

### CROSS-REFERENCE TO RELATED APPLICATIONS

The present application is a continuation application of International Application No. PCT/JP2017/036539, filed Oct. 6, 2017, which claims priority to Japanese Patent Application No. 2016-252951, filed Dec. 27, 2016. The contents of these applications are incorporated herein by reference in their entirety.

### FIELD

The present invention relates to a measurement device, a measurement method, and a non-transitory computer-readable recording medium.

### BACKGROUND

According to “Aortic dissection and aortic aneurysm guidelines (revised in 2011)” (“Guidelines for Diagnosis and Treatment of Aortic Aneurysm and Aortic Dissection”, (JCS 2011), JCS Joint Working Group, Circulation Journal Vol. 77, March 2013), an aortic aneurysm is defined as the case that a part of an aortic wall is locally expanded to form an aneurysm or the case that a diameter (outside diameter) is enlarged beyond a 1.5-fold (45 mm in a chest, 30 mm in an abdomen) of a normal diameter. The aortic aneurysm includes a fusiform aortic aneurysm as illustrated in FIG. 1A and a saccular aortic aneurysm as illustrated in FIG. 1B.

Conventionally, in order to evaluate these aneurysms, for example, Japanese Patent Application Publication No. 2013-94264 proposes a method in which the aneurysms are regarded as partial expansion of an elastic conduit, attention is paid to a loss of a pulse wave that transmits through the aneurysms, and presence or absence and/or a size of the aortic aneurysm is evaluated by obtaining the transfer function using pulse wave signals measured at two points on the arm and the leg.

### SUMMARY

According to a first aspect of the present disclosure, a measurement device that determines presence or absence of an abdominal aortic aneurysm in a subject, the measurement device includes
a pulse wave signal acquisition unit to acquire time-series pulse wave signals of each of an upper arm and an ankle of the subject,
a pulse wave velocity calculator to obtain a brachial-ankle pulse wave velocity based on the pulse wave signal of the upper arm and the pulse wave signal of the ankle,
a transfer function calculator to calculate a transfer function from the pulse wave signal of the upper arm and the pulse wave signal of the ankle so as to produce at least a phase diagram,
a phase diagram classifier to classify the phase diagram of each subject into any one of four groups, the phase diagram classifier setting a brachial-ankle pulse wave velocity (baPWV) line representing a inclined phase delay corresponding to the brachial-ankle pulse wave velocity on a frequency-phase plane on which the phase diagram is represented, and classifying the phase diagram of each subject into
a first group in which the phase diagram is along the baPWV line,
a second group in which the phase diagram is gradually separated from the baPWV line with increasing frequency,
a third group in which the phase diagram is separated from the baPWV line stepwise with increasing frequency, and
a fourth group in which with increasing frequency, the phase diagram is once separated from the baPWV line and comes close to the baPWV line again, and
an aneurysm determinator to determine the presence or absence of the abdominal aortic aneurysm by a criterion that is set according to each group with respect to each subject whose phase diagram is classified into any one of the four groups.

According to a second aspect of the present disclosure, a measurement method for determining presence or absence of an abdominal aortic aneurysm in a subject, the measurement method includes
acquiring time-series pulse wave signals of each of an upper arm and an ankle of the subject,
obtaining a brachial-ankle pulse wave velocity based on the pulse wave signal of the upper arm and the pulse wave signal of the ankle,
calculating a transfer function from the pulse wave signal of the upper arm and the pulse wave signal of the ankle so as to produce a gain diagram and a phase diagram,
classifying the phase diagram of each subject into any one of four groups, setting a brachial-ankle pulse wave velocity (baPWV) line representing a inclined phase delay corresponding to the brachial-ankle pulse wave velocity on a frequency-phase plane on which the phase diagram is represented, and classifying the phase diagram of each subject into
a first group in which the phase diagram is along the baPWV line,
a second group in which the phase diagram is gradually separated from the baPWV line with increasing frequency,
a third group in which the phase diagram is separated from the baPWV line stepwise with increasing frequency, and
a fourth group in which with increasing frequency, the phase diagram is once separated from the baPWV line and comes close to the baPWV line again, and
determining the presence or absence of the abdominal aortic aneurysm by a criterion that is set according to each group with respect to each subject whose phase diagram is classified into any one of the four groups.

According to a third aspect of the present disclosure, a non-transitory computer-readable recording medium storing program which, when executed by a computer, causes the computer to perform a measurement method for determining presence or absence of an abdominal aortic aneurysm in a subject, the measurement method includers
acquiring time-series pulse wave signals of each of an upper arm and an ankle of the subject,
obtaining a brachial-ankle pulse wave velocity based on the pulse wave signal of the upper arm and the pulse wave signal of the ankle,
calculating a transfer function from the pulse wave signal of the upper arm and the pulse wave signal of the ankle so as to produce a gain diagram and a phase diagram,
classifying the phase diagram of each subject into any one of four groups, setting a brachial-ankle pulse wave velocity (baPWV) line representing a inclined phase delay corresponding to the brachial-ankle pulse wave velocity on a frequency-phase plane on which the phase diagram is represented, and classifying the phase diagram of each subject into
a first group in which the phase diagram is along the baPWV line,
a second group in which the phase diagram is gradually separated from the baPWV line with increasing frequency,
a third group in which the phase diagram is separated from the baPWV line stepwise with increasing frequency, and
a fourth group in which with increasing frequency, the phase diagram is once separated from the baPWV line and comes close to the baPWV line again, and
determining the presence or absence of the abdominal aortic aneurysm by a criterion that is set according to each group with respect to each subject whose phase diagram is classified into any one of the four groups.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings.

FIG. 1A is a view schematically illustrating a fusiform aortic aneurysm.

FIG. 1B is a view schematically illustrating a saccular aortic aneurysm.

FIG. 2 is a view illustrating a pulse wave propagation model of systemic arteries and a vicinity of an abdominal aortic aneurysm in closeup.

FIG. 3A is a view illustrating an external appearance of a blood pressure pulse wave inspection device used to acquire clinical data.

FIG. 3B is a view illustrating a block configuration of the blood pressure pulse wave inspection device in a state in which a cuff is attached to a subject.

FIG. 4 is a diagram illustrating waveforms of pulse wave signals obtained from an upper right arm, an upper left arm, a right ankle, and a left ankle of a subject.

FIG. 5 is a view illustrating a flowchart of a measurement method according to an embodiment of the present invention.

FIG. 6 is a view illustrating a time-series pulse wave signal and one processing block obtained by dividing the pulse wave signal.

FIG. 7A is a view illustrating an example in which a phase diagram of one subject is classified into a first group.

FIG. 7B is a view illustrating an example in which a phase diagram of one subject is classified into a second group.

FIG. 7C is a view illustrating an example in which a phase diagram of one subject is classified into a third group.

FIG. 7D is a view illustrating an example in which a phase diagram of one subject is classified into a fourth group.

FIG. 8A is a view illustrating a first parameter for determining presence or absence of the abdominal aortic aneurysm.

FIG. 8B is a view illustrating a second parameter for determining the presence or absence of the abdominal aortic aneurysm.

FIG. 8C is a view illustrating a third parameter for determining the presence or absence of the abdominal aortic aneurysm.

FIG. 9A is a view illustrating a fourth parameter for determining the presence or absence of the abdominal aortic aneurysm.

FIG. 9B is a view illustrating a fourth parameter for determining the presence or absence of the abdominal aortic aneurysm.

FIG. 10 is a view illustrating a decision tree for determining the presence or absence of the abdominal aortic aneurysm, and including a view illustrating a criterion for a subject whose phase diagram is classified into the first group, views illustrating criteria for subjects whose phase diagrams are classified into the second and third groups, and views illustrating a criterion for a subject whose phase diagram is classified into the fourth group.

FIG. 11 is a view illustrating sensitivity and specificity.

FIG. 12A is a view illustrating detection rates for each of outer diameters (maximum minor axes) of the abdominal aortic aneurysm in forms of a bar graph.

FIG. 12B is a view illustrating detection rates for each of outer diameters (maximum minor axes) of the abdominal aortic aneurysm in forms of a table.

FIG. 13A is a view illustrating the detection rates for each of aneurysm shapes (fusiform, saccular) in forms of a bar graph.

FIG. 13B is a view illustrating the detection rates for each of aneurysm shapes (fusiform, saccular) in forms of a table.

FIG. 14 is a view illustrating a scatter diagram of the outer diameter (maximum minor axis) of the aneurysm and (measured) baPWV of AAA patients.

FIG. 15 is a view illustrating a block configuration of a measurement device according to an embodiment of the present invention.

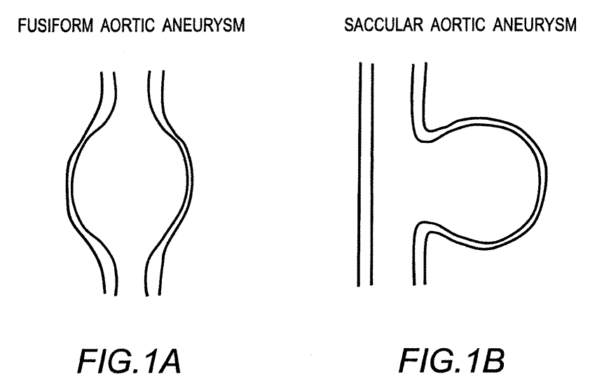

### DETAILED DESCRIPTION

The embodiments will now be described with reference to the accompanying drawings, wherein like reference numerals designate corresponding or identical elements throughout the various drawings.

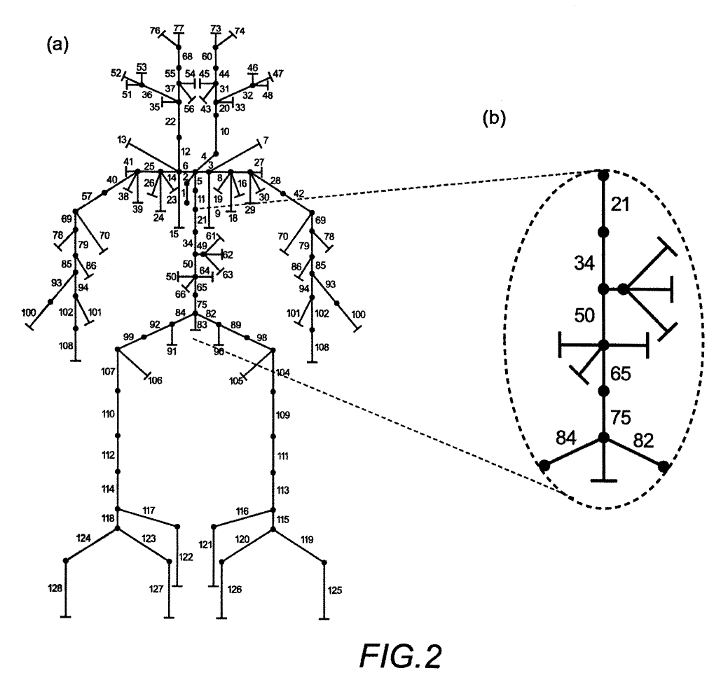

An artery diameter of a pulse wave propagation model used for verification in Japanese Patent Application Publication No. 2013-94264 ((a) in FIG. 2 illustrates a pulse wave propagation model of a whole body artery, and (b) in FIG. 2 illustrates an enlarged portion in the vicinity of the abdominal aorta) is smaller than that of an actual living body. Additionally, because the inside diameter of the abdominal artery that reproduces the Abdominal Aortic Aneurysm (AAA) is set to 100 mm that is larger than or equal to three times that of the actual abdominal aortic aneurysm and verification is performed, there is a possibility that a change in a transfer function such as a transmission loss is overestimated. As described above, the method of Japanese Patent Application Publication No. 2013-94264 deviates from clinical data, and there is a question about evaluation accuracy.

Hereinafter, embodiments of the present invention will be described in detail with reference to the drawings.

### (1) Construction of Case Database Used for Construction and Verification of Algorithm

A case database used for construction and verification of an algorithm is constructed as follows.

### (1-1) Data Acquisition Device

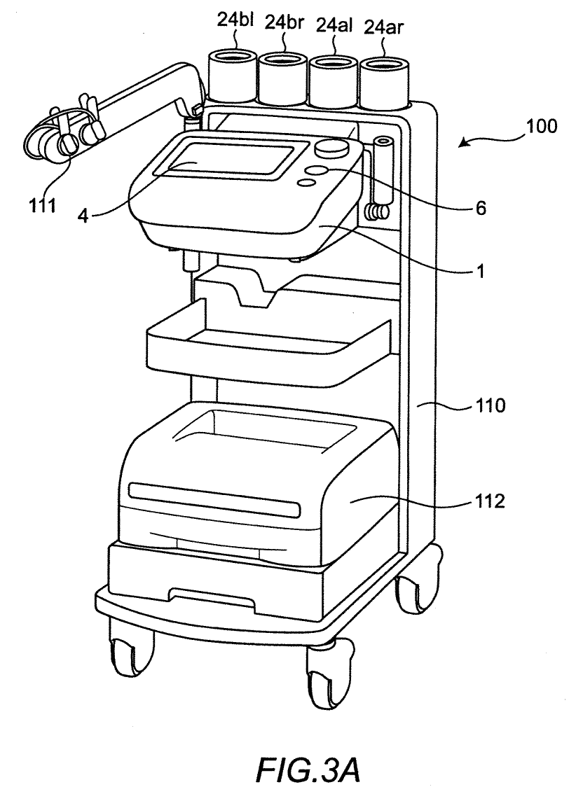

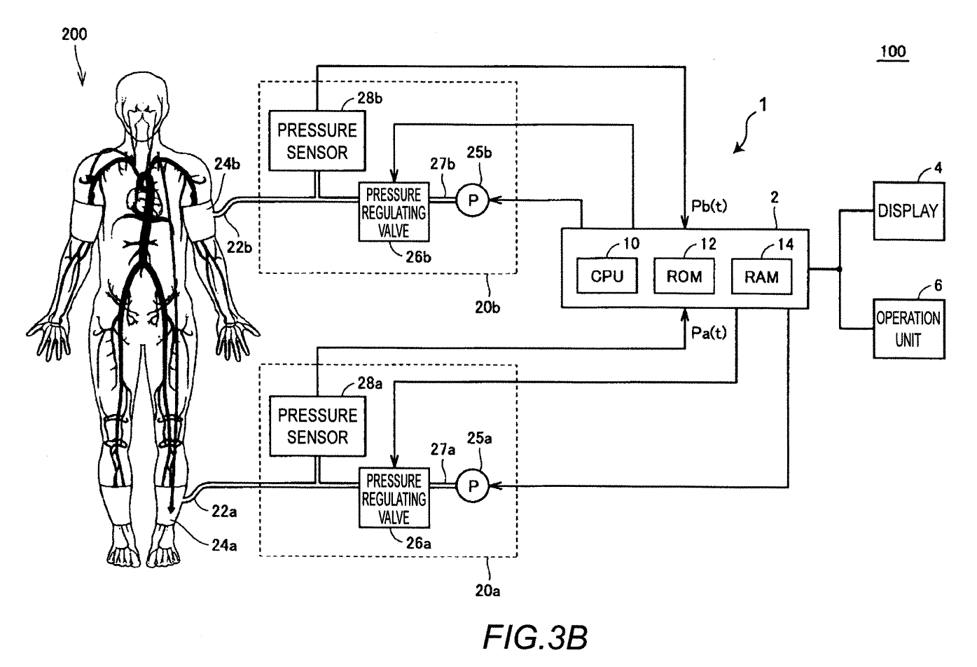

Pulse wave signals of the upper arm and a leg of a subject are acquired by a blood pressure pulse wave inspection device (BP203RPEIII Form 3 or BP-203RPEII Form 2: manufactured by Omron Choline Co., Ltd. (Tokyo, Japan)) (represented by reference numeral 100). As illustrated in FIG. 3A, the device 100 includes a main body 1 mounted on a stand 110, four cuffs 24ar, 24a1, 24br, 24b1, a heart sound or electrocardiogram measurement tool 111, and a printer 112. A display 4 and an operation unit 6 are provided in the main body 1. The four cuffs 24ar, 24a1, 24br, 24b1 are designed to be attached to a right ankle, a left ankle, an upper right arm, and an upper left arm of the subject, respectively. In the following description, the cuffs 24ar, 24a1 for the right and left ankles are collectively referred to as a cuff 24a, and the cuffs 24br and 24b1 for the upper right and upper left arms are collectively referred to as a cuff 24b.

FIG. 3B illustrates a block configuration of the main body 1 of the device 100 while the cuff 24a and the cuff 24b are attached to the ankles and the upper arms of a subject 200. The main body 1 includes a processor 2 and measurement units 20a, 20b in addition to the display 4 and the operation unit 6. For convenience, the measurement units 20a, 20b only for the left ankle and the upper left arm are illustrated in the figure, but measurement units for the right ankle and the upper right arm are similarly provided.

The processor 2 controls the entire device 100. Typically, the processor 2 is constructed with a computer including a central processing unit (CPU) 10, a read only memory (ROM) 12, and a random access memory (RAM) 14.

The CPU 10 reads a program previously stored in the ROM 12, and executes the program using the RAM 14 as a work memory.

The display 4, the operation unit 6, and the printer 112 in FIG. 3A are connected to the processor 2. The display 4 encourages a user to input various settings, and displays a calculation result from the processor 2. On the other hand, the user operates the operation unit 6 while checking contents displayed on the display 4, and performs desired setting input and operation. In this example, the display 4 is constructed with a light emitting diode (LED), a liquid crystal display (LCD), or the like. The printer 112 prints out the calculation result and the like displayed on the display 4 onto paper.

More specifically, the processor 2 in FIG. 3B provides a measurement command to the measurement units 20a, 20b, receives measurement signals Pa(t), Pb(t) measured in response to the measurement commands, and acquires clinical data (to be described later) based on the measurement signals Pa(t), Pb(t).

The measurement units 20a, 20b pressurize an internal pressure (hereinafter, referred to as a “cuff pressure”) of cuffs (air bladders) 24a, 24b attached to a predetermined measurement region of the subject 200, and measure a time waveform of a pulse wave at each measurement region. That is, the measurement signals Pa(t), Pb(t) become pulse wave signals at positions where the cuffs 24a, 24b are attached, respectively. Because the processor 2 executes processing using a frequency characteristic between the measurement signals Pa(t), Pb(t), a measurement command is provided from processor 2 to the measurement units 20a, 20b such that the measurement units 20a, 20b can measure the measurement signals in synchronization with each other.

More specifically, for example, the cuffs 24a, 24b are attached to the ankle (preferably, around the anterior tibial artery) and the upper arm (preferably, around the brachial artery) of the subject 200, and pressurized by air supplied from the measurement units 20a, 20b through pipes 22a, 22b, respectively. The cuffs 24a, 24b are pressed against the corresponding measurement regions by the pressurization, and pressure changes corresponding to the pulse waves of the measurement regions are transmitted to the measurement units 20a, 20b through the pipes 22a, 22b, respectively.

The measurement units 20a, 20b measure the time waveforms of the pulse waves in the measurement regions by detecting the transmitted pressure changes. Because preferably arithmetic processing is performed on a predetermined frequency component (for example, 0 to 20 [Hz]) of the measurement signals Pa(t), Pb(t), preferably a measurement cycle (sampling cycle) of the measurement signals Pa(t), Pb(t) is shorter than a time interval (for example, 25 msec) corresponding to the frequency component.

In order to perform the measurement operation, the measurement unit 20a includes a pressure sensor 28a, a pressure regulating valve 26a, a pressure pump 25a, and a pipe 27a. The pressure sensor 28a detects a pressure fluctuation transmitted through the pipe 22a. The pressure sensor 28a includes a plurality of sensor elements arrayed at predetermined intervals on a semiconductor chip such as single-crystal silicon. The pressure regulating valve 26a is interposed between the pressure pump 25a and the cuff 24a, and maintains the pressure used to pressurize the cuff 24a in a predetermined range during a measurement time. The pressure pump 25a operates in response to the measurement command from the processor 2, and supplies pressurized air in order to pressurize the cuff 24a.

Similarly, the measurement unit 20b includes a pressure sensor 28b, a pressure regulating valve 26b, a pressure pump 25b, and a pipe 27b. The configuration of each unit is similar to that of the measurement unit 20a.

### (1-2) Acquisition of Clinical Data

The blood pressure values (Systolic Blood Pressure (SBP) and Diastolic Blood Pressure (DBP)) of the four limbs of the subject 200 are simultaneously measured using the device 100, an Ankle Brachial Index (ABI) is calculated, the pulse wave signals of the four limbs are acquired for a fixed time at a predetermined cuff pressure after the blood pressure is measured, and brachial-ankle Pulse Wave Velocity (baPWV) is acquired.

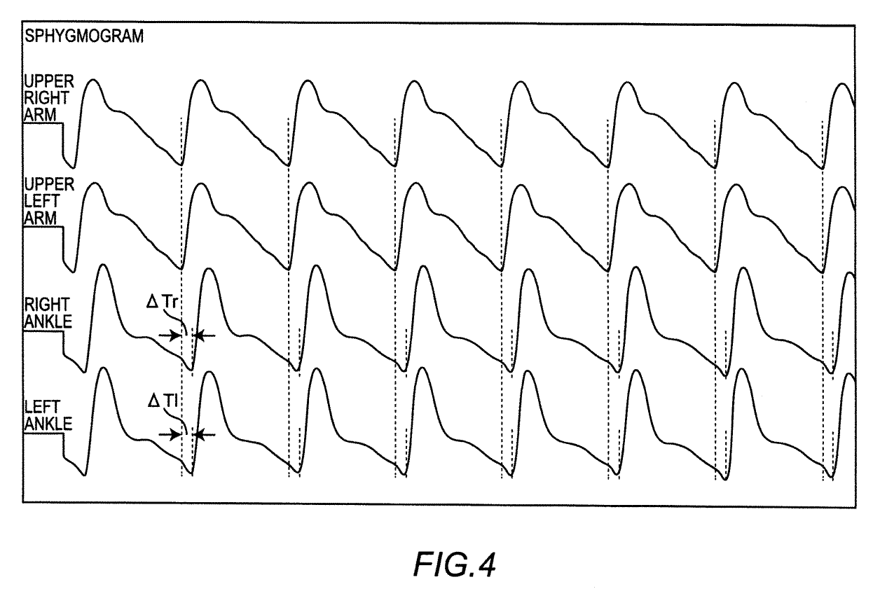

For example, based on delays ΔT1, ΔTr of a rising phase in the pulse wave signal in FIG. 4 (in this example, rising delays of the waveform of the ankle to the rising phase of the waveform of the upper arm become ΔT1, ΔTr for left and right half bodies of the subject 200), the brachial-ankle pulse wave velocity baPWV is calculated for the left and right half bodies of the subject 200 by baPWV=(Lb−La)/ΔT. At this point, Lb represents a distance from an aortic starting portion to the ankle, and La represents a distance from the aortic starting portion to the upper arm. AT represents ΔT1 or ΔTr (for convenience, the symbols “1” and “r” are omitted).

### (2) Construction of Algorithm

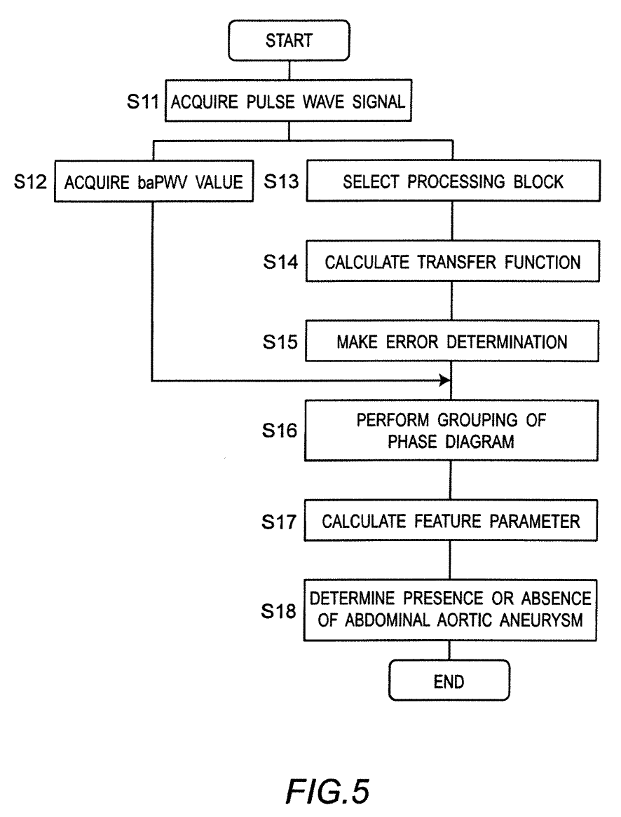

In order to determine presence or absence of the abdominal aortic aneurysm (AAA), a new algorithm is constructed as follows based on the clinical data such as the brachial-ankle pulse wave velocity baPWV and a phase diagram. FIG. 5 illustrates a flowchart determining the presence or absence of the abdominal aortic aneurysm including the acquisition of the pulse wave signal (step S11) and the acquisition of the brachial-ankle pulse wave velocity baPWV (step S12) as a flowchart of a measurement method of one embodiment.

### (2-1) Selection of Processing Block

A time-series pulse wave signal is temporally divided into a plurality of processing blocks, and the processing block to be used to determine the presence or absence of the abdominal aortic aneurysm is selected (Step S13 in FIG. 5).

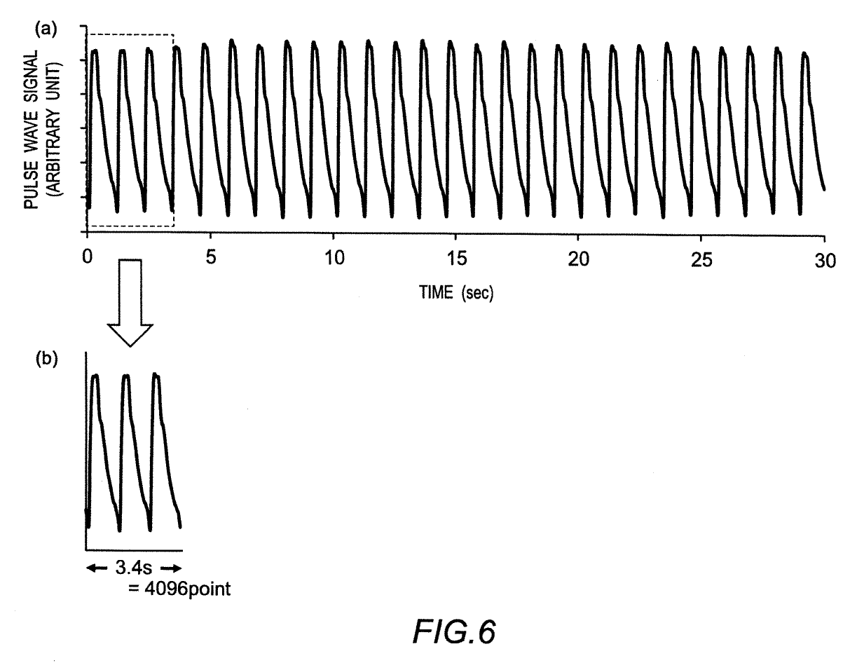

That is, in the pulse wave signal that is a biological signal, a pulse wave interval and an amplitude fluctuate every single pulse. When a transfer function is calculated using all the acquired pulse wave signal data, there is a possibility that a fluctuation component of the pulse wave signal included in the phase diagram adversely affects accuracy of the determination of the presence or absence of the abdominal aortic aneurysm. For this reason, the time-series pulse wave signal shown in (a) in FIG. 6 is divided into the plurality of processing blocks while shifted by a half of one processing block with about 3.4 s (seconds) shown in (b) in FIG. 6 as one processing block.

From the plurality of processing blocks obtained in this way, the processing block to be used to determine the presence or absence of the abdominal aortic aneurysm is selected as follows. A power spectrum Sxx is calculated for each processing block obtained from the pulse wave signal of the upper arm, and a fundamental frequency and a first harmonic are extracted. At the same time, a power spectrum Syy is calculated for each processing block obtained from the pulse wave signal of the ankle (leg), and the fundamental frequency and the first harmonic are extracted. Subsequently, the processing block having the largest number of identical fundamental frequencies and first harmonics is extracted in the upper arm. The processing block of the ankle (leg) exhibiting the fundamental frequency and the first harmonic matched with the fundamental frequency and the first harmonic is selected as the processing block to be used to determine the presence or absence of the abdominal aortic aneurysm.

### (2-2) Calculation of Transfer Function

Subsequently, the transfer function is calculated from the pulse wave signal of the upper arm and the pulse wave signal of the ankle, and a gain diagram and a phase diagram are produced (step S14 in FIG. 5).

In calculating the transfer function, in order to reduce an influence of noise, the power spectrum Sxx of the upper arm and a cross spectrum Sxy of the upper arm and the leg are calculated for each processing block, and an arithmetic mean is calculated with respect to the plurality of processing blocks. A transfer function G is calculated by the following equation (Eq. 1) to produce the gain diagram and the phase diagram using a power spectrum Sxxave of the upper arm and a cross spectrum Sxyave of the upper arm and the leg, the power spectrum Sxxave and the cross spectrum Sxyave being obtained by the calculation of the arithmetic mean.

G=Sxyave/Sxxave (Eq. 1)

### (2-3) Error Determination

Subsequently, the data corresponding to any one of the following i) and ii) among the data of the learning group is excluded as an error that is data in which the presence or absence of the aortic aneurysm cannot correctly be detected (step S15 in FIG. 5).

### i) Data Suspected of Stricture of Leg

A pulse pressure in the leg becomes smaller than that in the upper arm in the case that the subject develops a stricture of the leg, so that an amplitude ratio of the upper arm and the leg becomes larger with increasing harmonics wave in the case that an amplitude component of the pulse wave is viewed on a frequency axis. Therefore, in the gain diagram, the data in which the gain of the first harmonic is less than or equal to the gain of the fundamental frequency is excluded as an error that is data suspected of the stricture of the leg.

### ii) Data Suspected of Mixing of Irregular Pulse Wave

The case that the number of processing blocks after the selection of the processing block is less than or equal to 4 in all the 16 processing blocks (1 processing block or less for all the 4 processing blocks) and the case that beats of ±25% or more to the average pulse wave interval are included in the remaining processing blocks are excluded as an error that is data suspected of mixing of the irregular pulse wave. Consequently, the mixing of the irregular pulse waves that cannot be removed by the above processing block selection is removed.

### (2-4) Grouping of Phase Diagram

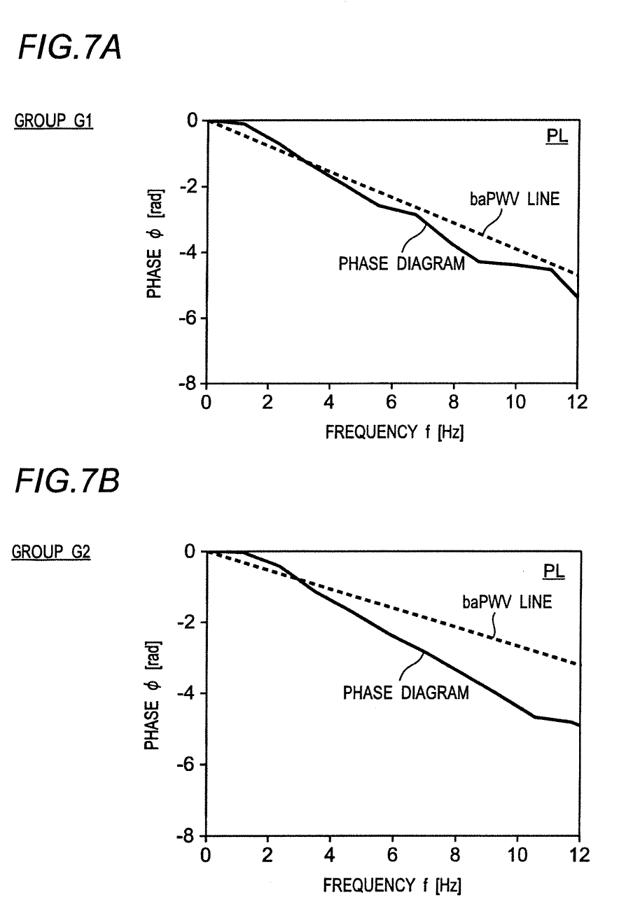

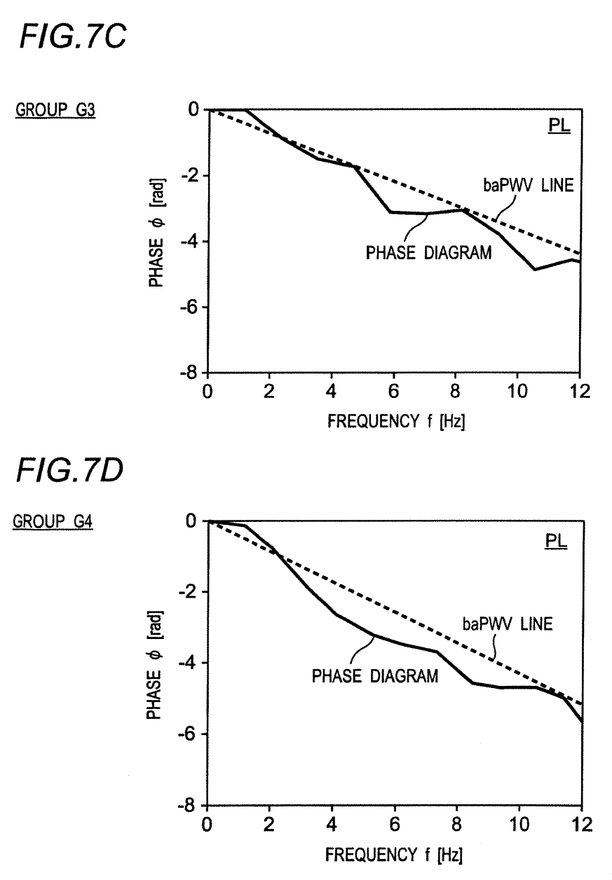

Subsequently, the phase diagram of each subject is classified into any one of four groups (step S16 in FIG. 5). This is because when the transfer function G of a learning group is calculated to calculate the phase diagram, there is a possibility that the shapes of the phase diagrams are roughly classified into four groups in FIGS. 7A to 7D.

In particular, as illustrated in FIG. 7A to 7D, a brachial-ankle pulse wave velocity baPWV line (represented by a broken line) representing a phase delay that is inclined according to the brachial-ankle pulse wave velocity baPWV is set on the frequency-phase plane PL where each phase diagrams (indicated by a solid line) is represented. In the baPWV line, the brachial-ankle pulse wave velocity (unit; m/s) is converted into units of the inclination on the frequency-phase plane PL (unit: deg/Hz), and is plotted on the plane PL as a line having the inclination and passing through an origin. Specifically, the inclination of the baPWV line is expressed by the following equation (Eq. 2), where the brachial-ankle distance is balength (unit; m) and the brachial-ankle pulse wave velocity is baPWV (unit; m/s).

−2π·balength/baPWV (Eq. 2)

In the example of FIG. 7A, the phase diagram is along the baPWV line. This type of data is classified into a first group G1. In the example of FIG. 7B, with increasing frequency f, the phase diagram is gradually separated from the baPWV line. This type of data is classified into a second group G2. In the example of FIG. 7C, with increasing frequency f increases, the phase diagram is separated stepwise from the baPWV line. This type of data is classified into the third group G3. In the example of FIG. 7D, with increasing frequency f increases, the phase diagram is once separated from the baPWV line, and comes close to the baPWV line again. This type of data is classified into the fourth group G4. Quantitatively, the data of the learning group is classified into the above four groups G1 to G4 according to a classification condition illustrated in Table 2. In Table 2, a phase difference between the baPWV line at a certain frequency f and the phase diagram is represented as Δφ.

TABLE 2
Classification condition of phase diagram

| | |
|---|---|
| First group | a) When f ≤ 6 Hz, Δφ takes a value larger than −0.55, |
| G1 | and |
| | b) When 6 Hz < f ≤ 10 Hz, Δφ takes a value larger than |
| | −0.95, and |
| | c) When f ≤ 12.20 Hz, a maximum value between peaks |
| | takes a value larger than or equal to −0.5, and |
| | d) When f ≤ 10 Hz, a minimum value of Δφ is a peak. |
| Second group | The above conditions a) or b) is satisfied, and |
| G2 | e) When (a frequency having a first peak at frequencies |
| | higher than 4 Hz) ≤ f ≤ 12.20 Hz, Δφ does not take a |
| | value larger than or equal to −0.5, and |
| | f) There is no frequency having a peak at frequencies |
| | larger than 4 Hz. |
| Third group | When (a frequency having the first peak at frequencies |
| G3 | higher than 4 Hz) ≤ f ≤ 12.20 Hz, there are two places |
| | where Δφ takes a value larger than or equal to −0.5 |
| | (when the peak is counted as one section). |
| Fourth group | When (a frequency having the peak first at frequencies |
| G4 | higher than 4 Hz) ≤ f ≤ 12.20 Hz, there is one place |
| | where Δφ has a value larger than or equal to −0.5 |
| | (when the peak is counted as one section). |

### (2-5) Calculation of Parameter

Subsequently, the following four parameters of a first parameter PR1, a second parameter PR2, a third parameter PR3, and a fourth parameter PR4 are calculate as a feature parameter representing a phenomenon specific to the data of the abdominal aortic aneurysm from the phase diagram and the gain diagram (step S17 in FIG. 5).

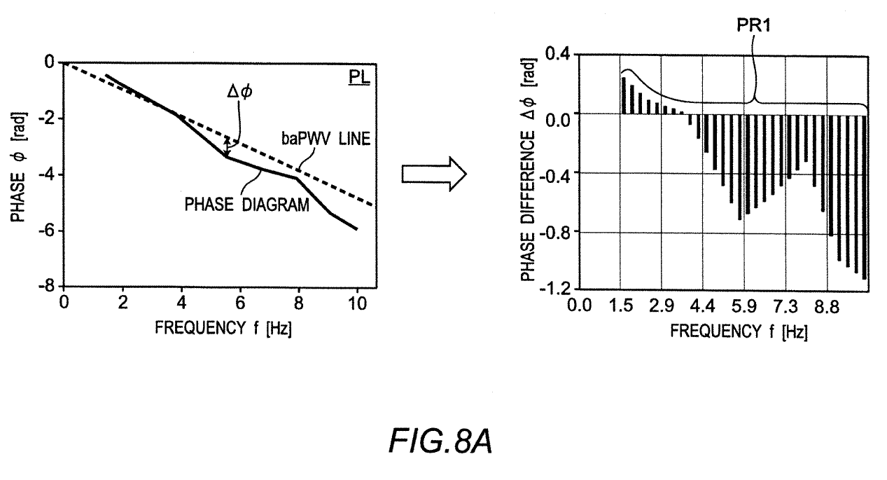

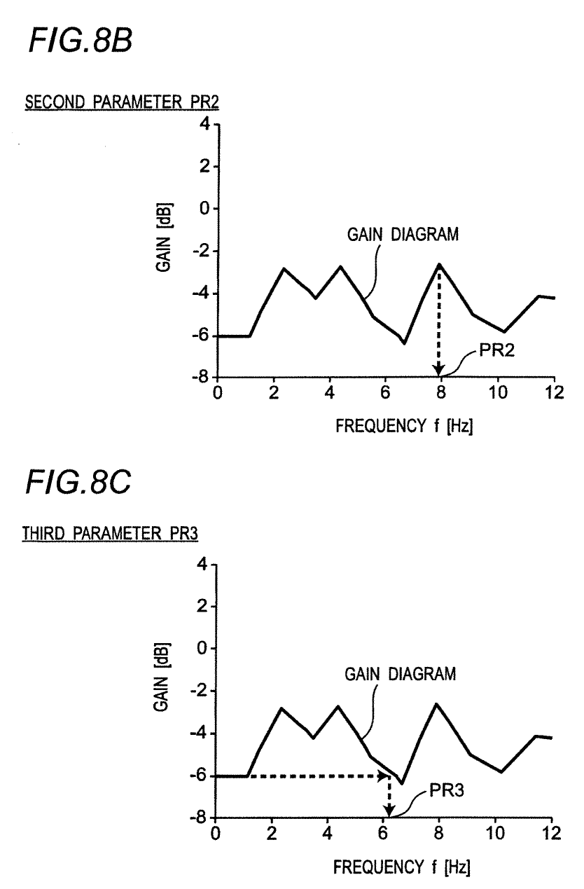

The first parameter PR1 is a sum of a difference between the phase diagram and the baPWV line. As disclosed in Japanese Patent Application Publication No. 2013-94264, the phase diagram changes with increasing inner diameter of the abdominal aortic aneurysm. An index that quantifies the amount of change is set to the first parameter PRE The first parameter PR1 becomes the index related to the inside diameter of the abdominal aortic aneurysm. Specifically, for example, as illustrated in FIG. 8A, when the phase diagram and the baPWV line are represented on the frequency-phase plane PL, the sum of the difference Asp (absolute value) between the baPWV line and the phase diagram in a predetermined target frequency range (in this example, the frequency f=1.47 Hz to 10.25 Hz) is set to the first parameter PR1. It can be said that the possibility of the abdominal aortic aneurysm increases with increasing value of the first parameter PRE

The second parameter PR2 is a frequency that gives the maximum amplitude value in the gain diagram. As disclosed in Japanese Patent Application Publication No. 2013-94264, a frequency interval that takes the minimum value of the phase diagram increases with increasing Young's modulus of the abdominal aorta. An index that quantifies the frequency interval is set to the second parameter PR2. The second parameter PR2 is the index related to artery extensibility that is a generation mechanism of the abdominal aortic aneurysm. Specifically, as illustrated in FIG. 8B, in the gain diagram, the frequency at which the gain becomes maximum in a predetermined target frequency range (in this example, frequency f=0 Hz to 8 Hz) is set to the second parameter PR2. It can be said that the possibility of the abdominal aortic aneurysm increases with increasing value of the second parameter PR2.

The third parameter PR3 is the frequency that gives the same gain as the gain of the fundamental frequency of the pulse wave signal in the gain diagram. As disclosed in Japanese Patent Application Publication No. 2013-94264, the frequency interval that takes the minimum value of the transfer function becomes narrower in proportion to a length of the abdominal aortic aneurysm. The index that quantifies the frequency interval is set to the third parameter PR3. The third parameter PR3 is the index related to the length of the abdominal aortic aneurysm. Specifically, as illustrated in FIG. 8C, in the gain diagram, the frequency which becomes the same gain of the fundamental frequency of the pulse wave signal in a predetermined target frequency range (in this example, the range from the fundamental frequency of the pulse wave signal to 10 Hz) is set to the third parameter PR3. In practice, because the gain diagram only takes values for each frequency resolution, the frequency immediately after falling below the value of the gain of the fundamental frequency of the pulse wave signal while searching from the low frequency side is set to the third parameter PR3. It can be said that the possibility of the abdominal aortic aneurysm increases with decreasing value of the third parameter PR3.

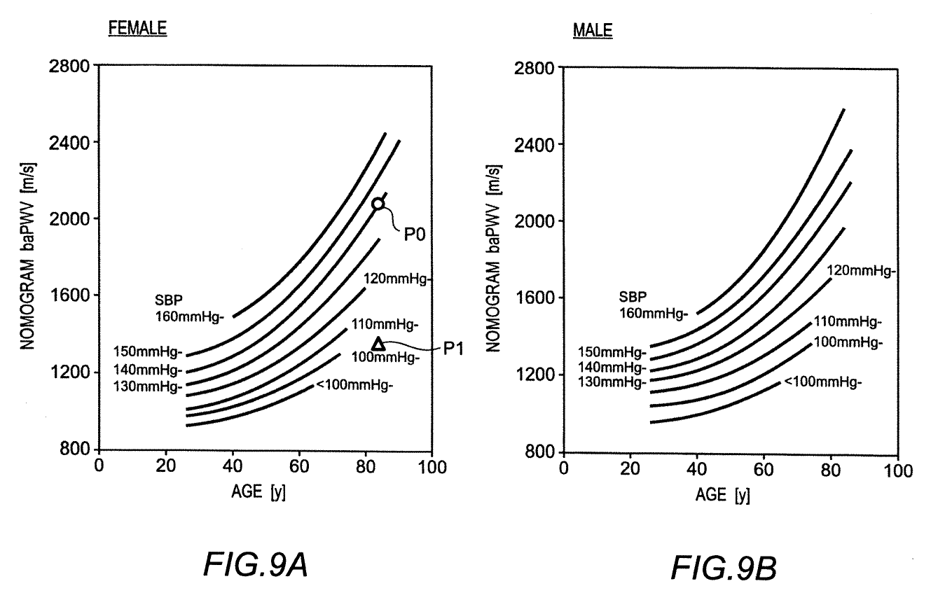

The fourth parameter PR4 is a difference AbaPWV between a statistical brachial-ankle pulse wave velocity (this is referred to as “nomogram guess baPWV”) obtained from a statistical chart (for example, a nomograms illustrated in FIGS. 9A and 9B) for healthy subjects of the same age, sexuality and blood pressure as the subject and the brachial-ankle pulse wave velocity (this is referred to as “measured baPWV”) measured for the subject. The fourth parameter PR4 is calculated by the following equation (Eq. 3).

$$PR 4 ≡ Δ baPWV = ( nomogram guess baPWV ) - ( measured baPWV ) \qquad ( Eq . 3 )$$

For example, it is assumed that the measured baPWV of an 84-year-old female subject having a systolic blood pressure (SBP) of 140 mmHg is 1340 m/s as indicated by a A mark P1 in FIG. 9A. In this case, the nomogram estimated baPWV for a healthy subject having the same age, sex, and blood pressure as the subject is 2100 m/s as indicated by a ◯ mark P0 in FIG. 9A. Thus, the fourth parameter PR4=ΔbaPWV=760 m/s is obtained. The fourth parameter PR4 is the index that represents two events in the abdominal aortic aneurysm. One represents arteriosclerosis (aortic extensibility) that is one of the generation mechanisms of the abdominal aortic aneurysms. The fourth parameter PR4 can take a positive value because the baPWV becomes faster as the arteriosclerosis progresses (the aortic extensibility decreases). On the other hand, according to Bramwell and Hill et al. (“Velocity of transmission of the pulse-Wave and elasticity of arteries”, Bramwell J C, Hill A V, Lancet, 1922; 199 (5149); 891-892), it is reported that carotid artery-radial artery PWV decreases in the patient of the aortic aneurysm. This is attributed to the fact that baPWV decreases by expansion of the aortic inner diameter due to the aortic aneurysm or a change of an aortic wall property. Consequently, the fourth parameter PR4 can take a negative value.

### (2-6) Production of Decision Tree and Determination of Presence or Absence of Abdominal Aortic Aneurysm

The four groups G1 to G4 and the four parameters PR4 are combined to produce a decision tree in which sensitivity and specificity of the abdominal aortic aneurysm detection becomes the best. That is, a criterion for determining the presence or absence of the abdominal aortic aneurysm is produced according to each of the groups G1 to G4. Consequently, the presence or absence of the abdominal aortic aneurysm is determined for each subject whose phase diagram is classified into any one of the four groups G1 to G4 according to the criterion corresponding to each of the groups G1 to G4 (step S18 in FIG. 5).

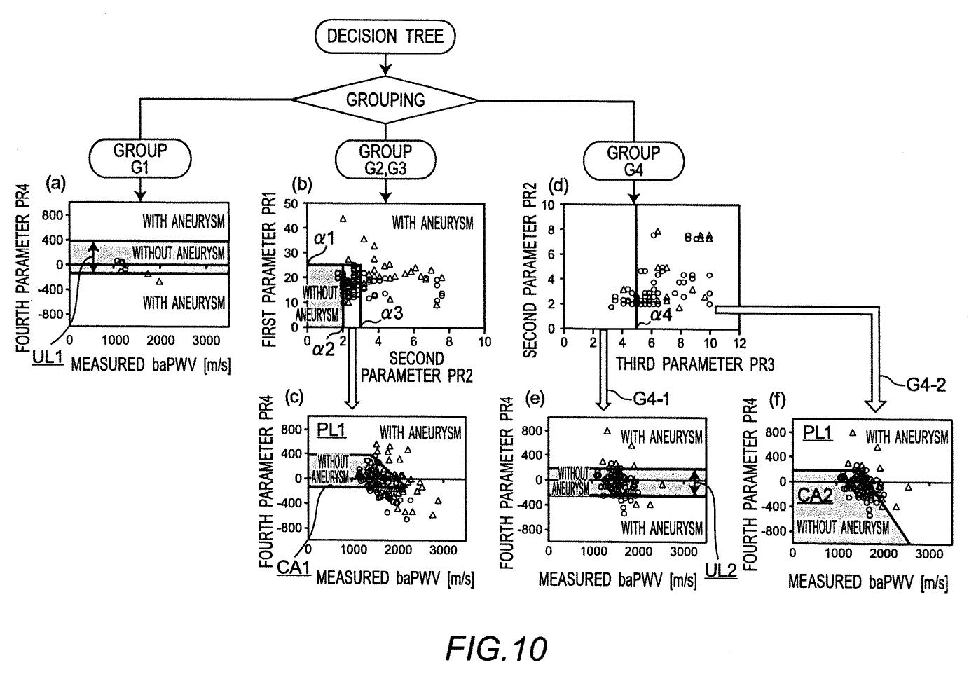

In particularly, an upper part of FIG. 10 illustrates four groups G1 to G4 classified by grouping of phase diagrams (step S16 of FIG. 5). In producing the decision tree, a scatter diagram by a combination of the first parameter PR1 and the second parameter PR2 and a scatter diagram by a combination of the second parameter PR2 and the third parameter PR3 are produced for each of the groups G1 to G4. A scatter diagram in which a horizontal axis is the measured baPWV is produced for the fourth parameter PR4. From these scatter diagrams, the decision tree and a threshold for the abdominal aortic aneurysm detection are constructed as follows for each of the groups G1 to G4.

### (2-6-1) Criterion for First Group G1

The first group G1 is a group having a low possibility of the abdominal aortic aneurysm because a significant change does not exist in the phase diagram. For this reason, as illustrated in (a) in FIG. 10, when the fourth parameter PR4 falls within a first upper and lower limit range UL1 (in this example, −141<PR4<375.5), it is determined that there is no abdominal aortic aneurysm. On the other hand, based on the fourth parameter PR4, when it is considered that the measured baPWV decreases due to the influence of the abdominal aortic aneurysm (in this example, PR4≥375.5), or when it is considered that the artery extensibility decreases due to the influence of the abdominal aortic aneurysm (in this example, PR4≤−141), it is determined that there is the abdominal aortic aneurysm.

### (2-6-2) Criterion for Second Group G2 and Third Group G3

The second group G2 and the third group G3 are a group having a high possibility of the abdominal aortic aneurysm because the significant change is seen in the phase diagram. For this reason, as illustrated in (b) in FIG. 10, when the first parameter PR1 is less than a first threshold value α1 (in this example, PR1<25) in which the change in the phase diagram is considered to be small, and when the second parameter PR2 is smaller than a second threshold α2 (in this example, PR2<2) in which the artery extensibility is considered not to be decreased, it is determined that there is no abdominal aortic aneurysm. On the other hand, when the change in the phase diagram is considered to be large based on the first parameter PR1 (in this example, PR1≥25), or when the artery extensibility is considered to be decreased based on the second parameter PR2 (in this example, PR2≥3 when a third threshold α3 (=3) larger than the second threshold α2 (=2) for the second parameter PR2 is set), it is determined that there is the abdominal aortic aneurysm.

When the second group G2 and the third group G3 do not correspond to any of the above cases (PR1<25 and 2 PR2<3), as illustrated in (c) in FIG. 10, it is determined that there is no abdominal aortic aneurysm when a data point (indicated by the ◯ mark or the A mark in FIG. 10) defined by the fourth parameter PR4 and the measured baPWV falls within a first allowable region CA1 on a parameter plane PL1 in which the fourth parameter PR4 and the measured baPWV are axes orthogonal to each other. At this point, the first allowable region CA1 is defined as a region that is within the range of −260<PR4<170 with respect to the fourth parameter PR4 and is less than a threshold Th1 decided by the following expression (Eq. 4).

Th1=−0.7261×(measured baPWV)+1384 (Eq. 4)

The introduction of the equation (Eq. 4) has the following reason. That is, because the second group G2 and the third group G3 have a high probability of the abdominal aortic aneurysm, not only the value of the fourth parameter PR4 but also a relationship with the measured baPWV are desirably considered. On the other hand, when the data point decided by the fourth parameter PR4 and the measured baPWV is out of the first allowable region CA1 on the parameter plane PL1, it is determined that there is the abdominal aortic aneurysm.

### (2-6-3) Criterion for Fourth Group G4

The fourth group G4 is a group having a high possibility of the abdominal aortic aneurysm because the significant change is seen in the phase diagram. As illustrated in (d) in FIG. 10, the fourth group G4 is classified into a first sub-group G4-1 and a second sub-group G4-2 based on whether the third parameter PR3 is less than or equal to a fourth threshold α4 (=5).

The first sub-group G4-1 is a group in which the possibility of the presence of abdominal aortic aneurysm is increased because the third parameter PR3 is less than or equal to the fourth threshold α4 (in this example, PR3≤5). As illustrated in (e) in FIG. 10, for the first sub-group G4-1, it is determined that there is no abdominal aortic aneurysm when the fourth parameter PR4 falls within a second upper and lower limit range UL2 (in this example, −260<PR4<170), and it is determined that there is the abdominal aortic aneurysm when the fourth parameter PR4 is out of the second upper and lower limit range UL2 (that is, −260≥PR4 or 170≤PR4).

For the second sub-group G4-2 (that is, the data group satisfying 5<PR3), as illustrated in (f) in FIG. 10, when the data point (indicated by the ◯ mark or the A mark in FIG. 10) decided by the fourth parameter PR4 and the measured baPWV falls within a second allowable region CA2 on the parameter plane PL1 in which the axes of the fourth parameter PR4 and the measured baPWV are orthogonal to each other, it is determined that there is no abdominal aortic aneurysm. At this point, the second allowable region CA2 is defined as a region that is within the range of PR4<170 with respect to the fourth parameter PR4 and is less than a threshold Th2 decided by the following expression (Eq. 5).

Th2=−1.1093×(measured baPWV)+1804.7 (Eq. 5)

On the other hand, when the data point decided by the fourth parameter PR4 and the measured baPWV is out of the second allowable region CA2 on the parameter plane PL1, it is determined that there is the abdominal aortic aneurysm.

The presence or absence of the abdominal aortic aneurysm is determined as described above.

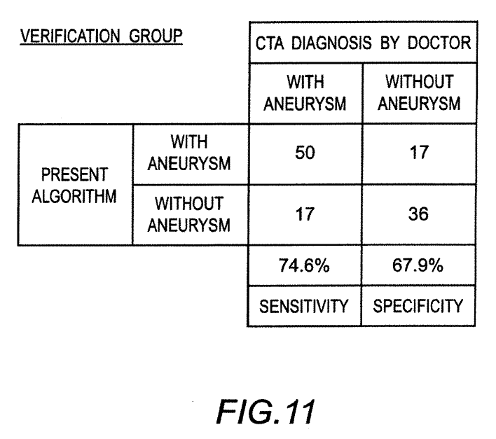

FIG. 11 illustrates the sensitivity and specificity of the determination result by the present algorithm for determining the presence or absence of the abdominal aortic aneurysm. In FIG. 11, a table side indicates a section determined to be “with aneurysm” and “without aneurysm” by the algorithm. A table head indicates a section determined to be “with aneurysm” and “without aneurysm” by CTA diagnosis (confirmed diagnosis) by a doctor. A table body indicates the number of cases applicable to those sections. As can be seen from FIG. 11, the sensitivity in a verification group is 74.6%, and the specificity is 67.9%.

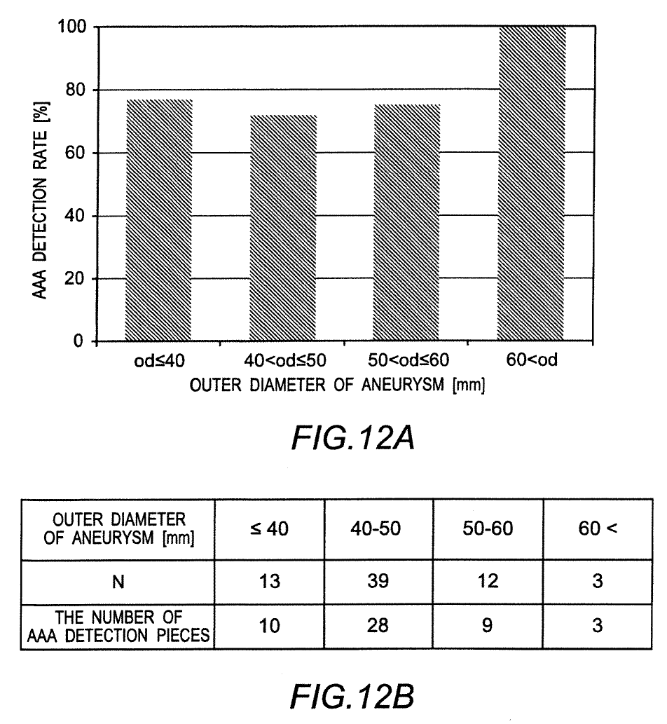

FIGS. 12A and 12B illustrate detection rates of each of outer diameters (maximum minor axes) of the abdominal aortic aneurysm in forms of a bar graph and a table, respectively. In these figures, the outer diameter of the aneurysm is divided into a range less than or equal to 40 mm, a range larger than 40 mm and less than or equal to 50 mm, a range larger than 50 mm and less than or equal to 60 mm, and a range larger than 60 mm. The detection rate is substantially constant in the three ranges except for the range larger than 60 mm Thus, according to the present algorithm, it can be said that the detection rate (determination result) of the abdominal aortic aneurysm does not depend on the outer diameter of the aneurysm.

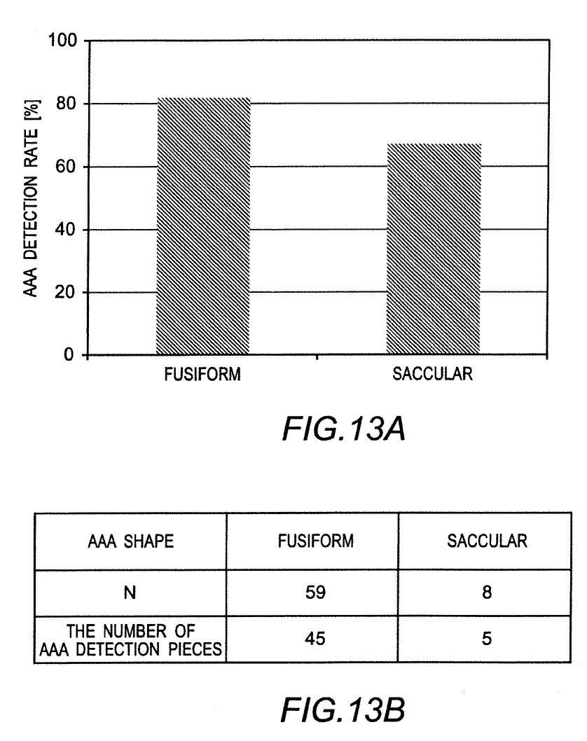

For reference, FIGS. 13A and 13B illustrate the detection rates for each of aneurysm shapes (fusiform, saccular) in forms of the bar graph and the table, respectively.

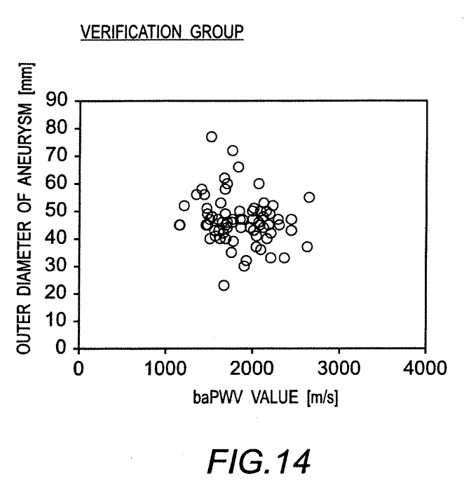

For reference, FIG. 14 illustrates a scatter diagram of the outer diameter (maximum minor axis) of the aneurysm and (measured) baPWV of AAA patients. As shown by Bailey et al. (“Carotid-femoral pulse wave velocity is negatively correlated with aortic diameter”, Bailey M A, Davies J M, Griffin K J, et al., Hypertension Research, 2014, 37, 926-932), there is no apparent correlation between the outer diameter (maximum short diameter) of the aneurysm and the baPWV.

As apparent from the above, according to the measurement method of the embodiment, the presence or absence of the abdominal aortic aneurysm in the subject can accurately be determined by the new algorithm based on the clinical data. The determination results of sensitivity of 74.6% and the specificity of 67.9% in the verification group (see FIG. 11) are considered to be a sufficiently beneficial level for primary screening in group medical checkup and the like.

### (Embodiment of Measurement Device)

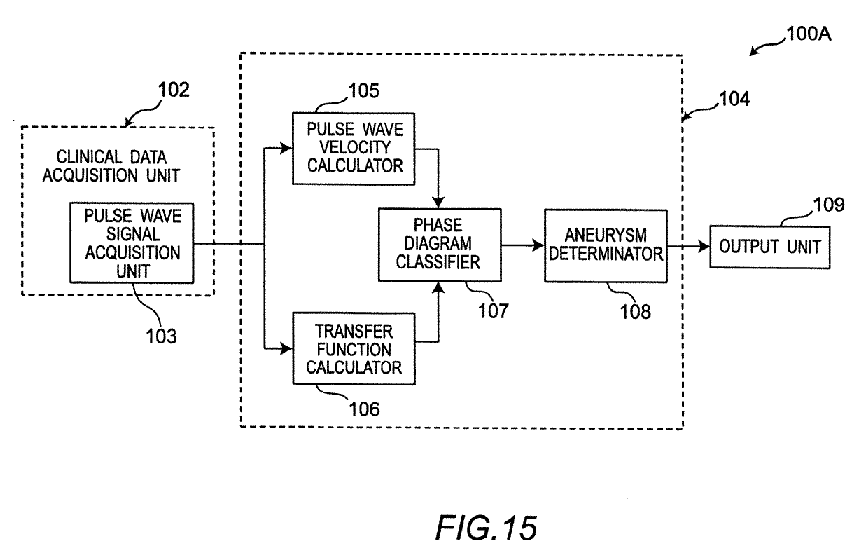

FIG. 15 illustrates a schematic block configuration of a measurement device (the whole is indicated by the reference numeral 100A) according to an embodiment of the present invention. The measurement device 100A is a measurement device that determines the presence or absence of the abdominal aortic aneurysm in the subject, and corresponds to one that performs the measurement method including the above new algorithm.

The measurement device 100A roughly includes a clinical data acquisition unit 102 (including a pulse wave signal acquisition unit 103), a signal processor 104, and an output unit 109.

The clinical data acquisition unit 102 simultaneously measures blood pressure values (systolic blood pressure (SBP) and diastolic blood pressure (DBP)) of the limbs of the subject, and the pulse wave signal acquisition unit 103 acquires the time-series pulse wave signals of the limbs for a fixed time at a predetermined cuff pressure (corresponds to step S11 in FIG. 5) after the measurement of the blood pressure. In this example, for example, the clinical data acquisition unit 102 includes the cuff 24a, the cuff 24b, the measurement units 20a, 20b controlled by the processor 2, and the pipes 22a, 22b that connect the cuffs 24a, 24b and the measurement units 20a, 20b as illustrated in FIG. 3B.

The signal processor 104 includes a pulse wave velocity calculator 105, a transfer function calculator 106, a phase diagram classifier 107, and an aneurysm determinator 108. The signal processor 104 is constructed with a computer including a central processing unit (CPU), a read only memory (ROM), and a random access memory (RAM).

The pulse wave velocity calculator 105 obtains the brachial-ankle pulse wave velocity baPWV based on the pulse wave signal of the upper arm and the pulse wave signal of the ankle that are acquired by the pulse wave signal acquisition unit 103 (corresponds to step S12 in FIG. 5).

The transfer function calculator 106 calculates the transfer function from the pulse wave signal of the upper arm and the pulse wave signal of the ankle that are acquired by the pulse wave signal acquisition unit 103, and produces at least the phase diagram as illustrated in FIGS. 7A to 7D (corresponds to step S14 in FIG. 5). Preferably the transfer function calculator 106 previously selects the processing block (corresponds to step S13 in FIG. 5). Desirably an error determination (corresponds to step S15 in FIG. 5) is made after the phase diagram is produced.

The phase diagram classifier 107 classifies the phase diagram of each subject into any one of four groups G1 to G4 (corresponds to step S16 in FIG. 5). Specifically, the phase diagram classifier 107 sets the brachial-ankle pulse wave velocity baPWV line representing the phase delay that is inclined according to the brachial-ankle pulse wave velocity baPWV on the frequency-phase plane PL in which the phase diagrams illustrated in FIGS. 7A to 7D are represented. The phase diagram classifier 107 classifies the phase diagram of each subject into
the first group G1 in which the phase diagram is along the baPWV line,
the second group G2 in which the phase diagram gradually separates from the baPWV line with increasing frequency f,
the third group G3 in which the phase diagram is separated stepwise from the baPWV line with increasing frequency f, and
the fourth group G4 in which with increasing frequency f, the phase diagram is once separated from the baPWV line and comes close to the baPWV line again. The classification conditions of the phase diagram in Table 2 are used in the classification.

The aneurysm determinator 108 determines the presence or absence of the abdominal aortic aneurysm for each subject whose phase diagram is classified into any one of the four groups G1 to G4 according to the criterion corresponding to each of the groups G1 to G4 (corresponds to step S18 in FIG. 5).

At this point, the determination criterion corresponding to each of the groups G1 to G4 have already been described with reference to FIG. 10. As described above, the four parameters PR1 to PR4 described with reference to FIGS. 8A to 8C and 9 are calculated based on the clinical data of the subject (corresponds to step S17 in FIG. 5), and the determination criterion is set using the four parameters PR1 to PR4.

The output unit 109 outputs the determination result of the presence or absence of the abdominal aortic aneurysm obtained by the aneurysm determinator 108. For example, the output unit 109 includes the display 4 or the printer 112 as illustrated in FIG. 3A.

According to the measurement device 100A, the presence or absence of the abdominal aortic aneurysm in the subject can accurately be determined by the new algorithm based on the clinical data such as the pulse wave signal of the upper arm and the pulse wave signal of the ankle, the brachial-ankle pulse wave velocity derived from the pulse wave signal of the upper arm and the pulse wave signal of the ankle, and the phase diagram.

In addition to the above processing, the signal processor 104 may perform processing of calculating the clinical data such as an ankle-brachial index ABI, a normalized pulse wave area percentage MAP, and an upstroke time UT. The output unit 109 may output the determination result of the presence or absence of the abdominal aortic aneurysm together with the clinical data such as the ankle-brachial index ABI, the normalized pulse wave area percentage MAP, and the upstroke time UT.

In the above embodiment, by way of example, the time-series pulse wave signal of each of the upper arm and the ankle of the subject is measured and acquired as the pressure change using the cuffs 24a, 24b. However, the present invention is not limited to the embodiment. For example, a minute constant current may be applied to the measurement region of the subject, and a voltage change generated by a change in impedance (bio-impedance) gererated according to the propagation of the pulse wave may be acquired as the pulse wave signal.

The time-series pulse wave signals of the upper arm and the ankle of the subject may be input and acquired from the outside of the measurement device 100A through a wired or wireless communication line (such as a network).

### Other Embodiments

A program which is used to perform the measurement method of the above embodiment can also be provided. The program is non-transitorily recorded in a computer-readable recording medium, such as a flexible disk, a compact disk-read only memory (CD-ROM), a ROM, a RAM and a memory card, which is attached to the computer, and the program can be provided as a program product. Alternatively, the program can be provided while non-temporarily recorded in a recording medium such as a hard disk built in the computer. The program can also be provided by download through a network. For example, the above measurement method can be performed when the program is installed in a computer (processor 2) of the blood pressure pulse wave inspection device (BP203RPEIII Form 3 or BP-203RPEII Form 2: manufactured by Omron Choline Co., Ltd. (Tokyo, Japan)).

The above embodiments are illustrative only, and various modifications can be made without departing from the scope of the present invention. The plurality of embodiments described above can be made independently, and the embodiments can also be combined. Although various features in different embodiments can independently be established, the features in different embodiments can also be combined.

According to a measurement device of the present embodiment of the invention,
the measurement device that determines presence or absence of an abdominal aortic aneurysm in a subject, includes
a pulse wave signal acquisition unit to acquire time-series pulse wave signals of each of an upper arm and an ankle of the subject,
a pulse wave velocity calculator to obtain a brachial-ankle pulse wave velocity based on the pulse wave signal of the upper arm and the pulse wave signal of the ankle,
a transfer function calculator to calculate a transfer function from the pulse wave signal of the upper arm and the pulse wave signal of the ankle so as to produce at least a phase diagram,
a phase diagram classifier to classify the phase diagram of each subject into any one of four groups, the phase diagram classifier setting a brachial-ankle pulse wave velocity (baPWV) line representing a inclined phase delay corresponding to the brachial-ankle pulse wave velocity on a frequency-phase plane on which the phase diagram is represented, and classifying the phase diagram of each subject into
a first group in which the phase diagram is along the baPWV line,
a second group in which the phase diagram is gradually separated from the baPWV line with increasing frequency,
a third group in which the phase diagram is separated from the baPWV line stepwise with increasing frequency, and
a fourth group in which with increasing frequency, the phase diagram is once separated from the baPWV line and comes close to the baPWV line again, and
an aneurysm determinator to determine the presence or absence of the abdominal aortic aneurysm by a criterion that is set according to each group with respect to each subject whose phase diagram is classified into any one of the four groups.

As used herein, the “baPWV line representing the phase delay inclined according to the brachial-ankle pulse wave velocity” on the frequency-phase plane means that the brachial-ankle pulse wave velocity (unit; m/s) is converted into units of the inclination (unit; deg/Hz) on the frequency-phase plane and is plotted on the plane as a line having the inclination and passing through an origin. Specifically, the inclination of the baPWV line is represented by −2π□balength/baPWV, where a brachial-ankle distance is balength (unit; m) and the brachial-ankle pulse wave velocity is baPWV (unit; m/s).

According to the measurement device of the present embodiment of the invention, the presence or absence of the abdominal aortic aneurysm in the subject can accurately be determined by the new algorithm based on the clinical data such as the pulse wave signal of the upper arm and the pulse wave signal of the ankle, the brachial-ankle pulse wave velocity derived from the pulse wave signal of the upper arm and the pulse wave signal of the ankle, and the phase diagram.

According to the measurement device of an embodiment,
the transfer function calculator produces a gain diagram in addition to the phase diagram, and
in order to decide the criterion, the aneurysm determinator calculates and uses
a first parameter representing a sum of differences between the baPWV line and the phase diagram in a predetermined target frequency range on the frequency-phase plane,
a second parameter representing a frequency giving an amplitude maximum value in the gain diagram,
a third parameter representing a frequency giving a gain identical to a gain of a fundamental frequency of the pulse wave signal in the gain diagram, and
a fourth parameter representing a difference between a statistical brachial-ankle pulse wave velocity obtained from a statistical chart for healthy subjects having age, sexuality and blood pressure identical to those of the subject and the brachial-ankle pulse wave velocity measured for the subject.

The “target frequency range” includes the fundamental frequency of the pulse wave signal on the lower side, and the upper side indicates a range up to about 10 Hz.

The “fundamental frequency” of the pulse wave signal means the lowest frequency that gives a peak in a power spectrum. Typically the fundamental frequency is about 1 Hz to about 1.5 Hz.

The “statistical chart” typically means a nomogram of the brachial-ankle pulse wave velocity with age, sexuality, and blood pressure as a parameter. In the following description, the term “brachial-ankle pulse wave velocity” means the brachial-ankle pulse wave velocity actually measured for the subject with respect to a statistical brachial-ankle pulse wave velocity.

According to the measurement device of the embodiment,
the criterion set according to the first group is a criterion set by which it is determined that there is no abdominal aortic aneurysm when the fourth parameter falls within a first upper and lower limit range which are predetermined and it is determined that there is the abdominal aortic aneurysm when the fourth parameter is out of the first upper and lower limit range,
the criterion set according to the second group and the third group is a criterion set by which
it is determined that there is no abdominal aortic aneurysm when the first parameter and the second parameter are less than a first threshold and a second threshold which are predetermined, respectively, and
it is determined that there is the abdominal aortic aneurysm when the first parameter is larger than or equal to the first threshold or when the second parameter is larger than or equal to a third threshold larger than the second threshold,
when the first parameter is less than the first threshold and when the second parameter is larger than or equal to the second threshold and less than the third threshold, it is determined that there is no abdominal aortic aneurysm when a data point defined by the fourth parameter and the brachial-ankle pulse wave velocity falls within a first allowable region, which is predetermined, on a parameter plane in which the fourth parameter and the brachial-ankle pulse wave velocity are axes orthogonal to each other, and it is determined that there is the abdominal aortic aneurysm when the data point is out of the first allowable region,
the fourth group is classified into a first sub-group and a second sub-group based on whether the third parameter is less than or equal to a fourth threshold which is predetermined,
the criterion set according to the first sub-group is a criterion set by which it is determined that there is no abdominal aortic aneurysm when the fourth parameter falls within a second upper and lower limit range which is predetermined and it is determined that there is the abdominal aortic aneurysm when the fourth parameter is out of the second upper and lower limit range, and
the criterion set according to the second sub-group is a criterion set by which it is determined that there is no abdominal aortic aneurysm when the data point defined by the fourth parameter and the brachial-ankle pulse wave velocity falls within a second allowable region, which is predetermined, on the parameter plane in which the fourth parameter and the brachial-ankle pulse wave velocity are axes orthogonal to each other and it is determined that there is the abdominal aortic aneurysm when the data point is out of the second allowable region.

According to another aspect of the present embodiment of the invention,
a measurement method for determining presence or absence of an abdominal aortic aneurysm in a subject, includes
a step of acquiring time-series pulse wave signals of each of an upper arm and an ankle of the subject,
a step of obtaining a brachial-ankle pulse wave velocity based on the pulse wave signal of the upper arm and the pulse wave signal of the ankle,
a step of calculating a transfer function from the pulse wave signal of the upper arm and the pulse wave signal of the ankle so as to produce a gain diagram and a phase diagram,
a step of classifying the phase diagram of each subject into any one of four groups, setting a brachial-ankle pulse wave velocity (baPWV) line representing a inclined phase delay corresponding to the brachial-ankle pulse wave velocity on a frequency-phase plane on which the phase diagram is represented, and classifying the phase diagram of each subject into
a first group in which the phase diagram is along the baPWV line,
a second group in which the phase diagram is gradually separated from the baPWV line with increasing frequency,
a third group in which the phase diagram is separated from the baPWV line stepwise with increasing frequency, and
a fourth group in which with increasing frequency, the phase diagram is once separated from the baPWV line and comes close to the baPWV line again, and
a step of determining the presence or absence of the abdominal aortic aneurysm by a criterion that is set according to each group with respect to each subject whose phase diagram is classified into any one of the four groups.

According to the measurement method of the present embodiment of the invention, the presence or absence of the abdominal aortic aneurysm in the subject can accurately be determined by the new algorithm based on the clinical data such as the pulse wave signal of the upper arm and the pulse wave signal of the ankle, the brachial-ankle pulse wave velocity derived from the pulse wave signal of the upper arm and the pulse wave signal of the ankle, and the phase diagram.

Either of the step of obtaining the pulse wave velocity and the step of calculating the transfer function may be performed first, or performed in parallel with each other.

According to still another aspect of the present embodiment of the invention, a program causes a computer to perform the measurement method.

According to the program of the present embodiment of the invention, the measurement method can be performed by a computer. Consequently, the presence or absence of the abdominal aortic aneurysm in the subject can accurately be determined.

Desirably the program is recorded in a computer-readable recording medium. More preferably the program is recorded in a non-transitory computer-readable recording medium. Consequently, the computer can read the program from the recording medium to perform the measurement method. As a result, the presence or absence of the abdominal aortic aneurysm in the subject can accurately be determined.

As apparent from the above, according to the measurement device, the measurement method, and the program of the present embodiment of the invention, the presence or absence of abdominal aortic aneurysm in the subject can accurately be determined by the new algorithm based on the clinical data.

The above embodiments are illustrative, and are modifiable in a variety of ways without departing from the scope of this invention. It is to be noted that the various embodiments described above can be appreciated individually within each embodiment, but the embodiments can be combined together. It is also to be noted that the various features in different embodiments can be appreciated individually by its own, but the features in different embodiments can be combined.

What is claimed is:

1. A measurement device that determines presence or absence of an abdominal aortic aneurysm in a subject, the measurement device comprising:
a pulse wave signal acquisition unit to acquire time-series pulse wave signals of each of an upper arm and an ankle of the subject;
a pulse wave velocity calculator to obtain a brachial-ankle pulse wave velocity based on the pulse wave signal of the upper arm and the pulse wave signal of the ankle;
a transfer function calculator to calculate a transfer function from the pulse wave signal of the upper arm and the pulse wave signal of the ankle so as to produce at least a phase diagram;
a phase diagram classifier to classify the phase diagram of each subject into any one of four groups, the phase diagram classifier setting a brachial-ankle pulse wave velocity (baPWV) line representing a inclined phase delay corresponding to the brachial-ankle pulse wave velocity on a frequency-phase plane on which the phase diagram is represented, and classifying the phase diagram of each subject into
a first group in which the phase diagram is along the baPWV line,
a second group in which the phase diagram is gradually separated from the baPWV line with increasing frequency,
a third group in which the phase diagram is separated from the baPWV line stepwise with increasing frequency, and
a fourth group in which with increasing frequency, the phase diagram is once separated from the baPWV line and comes close to the baPWV line again; and
an aneurysm determinator to determine the presence or absence of the abdominal aortic aneurysm by a criterion that is set according to each group with respect to each subject whose phase diagram is classified into any one of the four groups.

2. The measurement device according to claim 1, wherein the transfer function calculator produces a gain diagram in addition to the phase diagram, and
in order to decide the criterion, the aneurysm determinator calculates and uses
a first parameter representing a sum of differences between the baPWV line and the phase diagram in a predetermined target frequency range on the frequency-phase plane,
a second parameter representing a frequency giving an amplitude maximum value in the gain diagram,
a third parameter representing a frequency giving a gain identical to a gain of a fundamental frequency of the pulse wave signal in the gain diagram, and
a fourth parameter representing a difference between a statistical brachial-ankle pulse wave velocity obtained from a statistical chart for healthy subjects having age, sex and blood pressure identical to those of the subject and the brachial-ankle pulse wave velocity measured for the subject.

3. The measurement device according to claim 2, wherein
the criterion set according to the first group is a criterion set by which it is determined that there is no abdominal aortic aneurysm when the fourth parameter falls within a first upper and lower limit range which are predetermined and it is determined that there is the abdominal aortic aneurysm when the fourth parameter is out of the first upper and lower limit range,
the criterion set according to the second group and the third group is a criterion set by which
it is determined that there is no abdominal aortic aneurysm when the first parameter and the second parameter are less than a first threshold and a second threshold which are predetermined, respectively, and
it is determined that there is the abdominal aortic aneurysm when the first parameter is larger than or equal to the first threshold or when the second parameter is larger than or equal to a third threshold larger than the second threshold,
when the first parameter is less than the first threshold and when the second parameter is larger than or equal to the second threshold and less than the third threshold, it is determined that there is no abdominal aortic aneurysm when a data point defined by the fourth parameter and the brachial-ankle pulse wave velocity falls within a first allowable region, which is predetermined, on a parameter plane in which the fourth parameter and the brachial-ankle pulse wave velocity are axes orthogonal to each other, and it is determined that there is the abdominal aortic aneurysm when the data point is out of the first allowable region,
the fourth group is classified into a first sub-group and a second sub-group based on whether the third parameter is less than or equal to a fourth threshold which is predetermined,
the criterion set according to the first sub-group is a criterion set by which it is determined that there is no abdominal aortic aneurysm when the fourth parameter falls within a second upper and lower limit range which is predetermined and it is determined that there is the abdominal aortic aneurysm when the fourth parameter is out of the second upper and lower limit range, and
the criterion set according to the second sub-group is a criterion set by which it is determined that there is no abdominal aortic aneurysm when the data point defined by the fourth parameter and the brachial-ankle pulse wave velocity falls within a second allowable region, which is predetermined, on the parameter plane in which the fourth parameter and the brachial-ankle pulse wave velocity are axes orthogonal to each other and it is determined that there is the abdominal aortic aneurysm when the data point is out of the second allowable region.

4. A measurement method for determining presence or absence of an abdominal aortic aneurysm in a subject, the measurement method comprising:
acquiring time-series pulse wave signals of each of an upper arm and an ankle of the subject;
obtaining a brachial-ankle pulse wave velocity based on the pulse wave signal of the upper arm and the pulse wave signal of the ankle;
calculating a transfer function from the pulse wave signal of the upper arm and the pulse wave signal of the ankle so as to produce a gain diagram and a phase diagram;
classifying the phase diagram of each subject into any one of four groups, setting a brachial-ankle pulse wave velocity (baPWV) line representing a inclined phase delay corresponding to the brachial-ankle pulse wave velocity on a frequency-phase plane on which the phase diagram is represented, and classifying the phase diagram of each subject into
a first group in which the phase diagram is along the baPWV line,
a second group in which the phase diagram is gradually separated from the baPWV line with increasing frequency,
a third group in which the phase diagram is separated from the baPWV line stepwise with increasing frequency, and
a fourth group in which with increasing frequency, the phase diagram is once separated from the baPWV line and comes close to the baPWV line again; and
determining the presence or absence of the abdominal aortic aneurysm by a criterion that is set according to each group with respect to each subject whose phase diagram is classified into any one of the four groups.

5. A non-transitory computer-readable recording medium storing program which, when executed by a computer, causes the computer to perform a measurement method for determining presence or absence of an abdominal aortic aneurysm in a subject, the measurement method comprising:
acquiring time-series pulse wave signals of each of an upper arm and an ankle of the subject;
obtaining a brachial-ankle pulse wave velocity based on the pulse wave signal of the upper arm and the pulse wave signal of the ankle;
calculating a transfer function from the pulse wave signal of the upper arm and the pulse wave signal of the ankle so as to produce a gain diagram and a phase diagram;
classifying the phase diagram of each subject into any one of four groups, setting a brachial-ankle pulse wave velocity (baPWV) line representing a inclined phase delay corresponding to the brachial-ankle pulse wave velocity on a frequency-phase plane on which the phase diagram is represented, and classifying the phase diagram of each subject into
a first group in which the phase diagram is along the baPWV line,
a second group in which the phase diagram is gradually separated from the baPWV line with increasing frequency,
a third group in which the phase diagram is separated from the baPWV line stepwise with increasing frequency, and
a fourth group in which with increasing frequency, the phase diagram is once separated from the baPWV line and comes close to the baPWV line again; and
determining the presence or absence of the abdominal aortic aneurysm by a criterion that is set according to each group with respect to each subject whose phase diagram is classified into any one of the four groups.
